(19) United States
(12) Patent Application Publication
(10) Pub. No.: US 2026/0137375 A1
(43) Pub. Date: May 21, 2026

(54) DNA Collection

(71) Applicant: Roy Lee Marks, JR., McKeesport, PA (US)

(72) Inventor: Roy Lee Marks, JR., McKeesport, PA (US)

(21) Appl. No.: 19/120,550

(22) PCT Filed: Oct. 11, 2023
(86) PCT No.: PCT/US23/34942

§ 371 Date: Apr. 11, 2025

Related U.S. Application Data
(60) Provisional application No. 63/415,008, filed on Oct. 11, 2022.

(51) Int. Cl. A61B 10/00; A61B 5/11; C12Q 1/6806

(52) U.S. Cl. CPC A61B 10/0045; A61B 5/1112; A61B 10/0096; C12Q 1/6806; A61B 2560/0295

(57) ABSTRACT

A DNA collection system having: a DNA collection device including an elongated hollow housing having a first end 2024/081313 and an opposed second end, and a plurality of DNA collection chambers mounted in a row in the housing, adjacent to the first end, each of the DNA collection chambers including a DNA collection component, and the collection component of each of the DNA collection chambers sealed from the other DNA collection components of the other 1 63723-WO-PCT DNA collection chambers, wherein each of the DNA collection chambers is removably mounted to adjacent DNA collection members.

### CROSS REFERENCE TO RELATED APPLICATIONS

The present application claims the benefit of U.S. provisional patent application 63/415,008, filed Oct. 11, 2022, the contents of which are incorporated herein in their entirety.

### BACKGROUND OF THE INVENTION

This invention relates to collection of deoxyribonucleic acid (DNA), and more particularly to apparatuses and methods for collecting DNA.

Collection of DNA samples may have various uses. When collecting DNA, it is important to be able to collect the desired DNA without cross contamination. This may be particularly true at a crime scene or when investigating a sexual crime, where collection of DNA evidence without cross contamination is important. In such circumstances, it is also important to readily note where the DNA sample was taken and to transfer the DNA sample into storage for testing. Unfortunately, many devices and methods for collecting, tracking location of DNA taken and transfer to storage for testing are less than satisfactory for important DNA collection, such as at a crime scene.

### SUMMARY OF THE INVENTION

According to an aspect, the invention may comprise a DNA collection system having: a DNA collection device including an elongated hollow housing having a first end and an opposed second end, and a plurality of DNA collection chambers mounted in a row in the housing, adjacent to the first end, each of the DNA collection chambers including a DNA collection component, and the collection component of each of the DNA collection chambers sealed from the other DNA collection components of the other DNA collection chambers, wherein each of the DNA collection chambers is removably mounted to adjacent DNA collection members.

According to an aspect, the invention may comprise a DNA collection device including: a specimen collection cylinder that is hollow, with a first end and an opposed second end; and a collection mesh mounted to the specimen collection cylinder adjacent to the first end, wherein the collection mesh is treated with at least one of a TKI and a TSI.

According to an aspect, the invention may comprise DNA collection device comprising: an elongated probe stem having an end; and a probe tip assembly removably mounted to the end and including a hollow conically shaped DNA collection chamber having a sample hole at a narrow end spaced from the stem, a DNA collection component in the DNA collection chamber adjacent to the sample hole configured to collect a DNA sample through the sample hole.

According to an aspect, the invention may comprise A kit for collecting DNA having: a DNA collection device including: an elongated hollow housing having a first end; a master RFID tag mounted to the housing; and a DNA collection chamber removably mounted to the first end and including a DNA collection component mounted thereto, wherein the DNA collection component is treated with at least one of a TKI and a TSI.

According to an aspect, the invention may provide a DNA collection device that collects DNA specimens without risk of cross contamination of the DNA specimens. Such device may be useful in, for example, a crime scene investigation, a paternity investigation, a sexual crime investigation, or other investigations where DNA samples are helpful.

According to an aspect, the invention may provide a DNA collection device that allows for one or multiple (e.g., 10) DNA specimens to be taken (which may be essentially simultaneously) without cross contamination. The device may include multiple (e.g., 10) sealed chambers that each may take a separate DNA specimen(s) without cross contamination between any of the separate specimens taken. Each chamber may have a threaded (i.e., twist-off) connection or a snap-off connection for ease of removal from the DNA collection device after a DNA sample has been taken.

According to an aspect, the invention may provide a method for collecting and storing DNA samples.

According to an aspect, the invention may provide a method for marking where individual DNA samples were taken. The DNA collection device may include one or multiple (e.g., 10) specimen location markers corresponding to the individual specimen collection chambers. Marking location solution may identify DNA sample area with a non-DNA-contaminating solution. The specimen location markers may include a solution of, for example, tyrosinase inhibitor (TSI) or tyrosine kinase inhibitors (TKI) and chlorophyll for coloring, which may comprise a chlorophyll dye solution filled reservoir that is pressure activated to release at the outermost chamber.

According to an aspect, the solution, employing for example TSI or TKI, may act as a stabilizer or inhibitor that may be able to preserve DNA without contaminating the DNA sample. The stabilizer may help to preserve a DNA sample for a relatively long time (e.g., years). The stabilizer may be rubbed over an area where DNA is collected without contaminating the DNA sample.

According to an aspect, the invention may provide a DNA collection device that includes a master radio-frequency identification (RFID) chip as well as an RFID chip for each of the multiple DNA collection chambers of the device. The RFID chip may provide Global Positioning System (GPS) tracking, a real time specimen classification chip, other real time tracking, or a combination of these features. According to an aspect, the invention may provide for the RFID chip to store and transmit the collected information to two or more other locations (e.g., cloud storage, a criminal filing system, etc.) to provide backup for the information received on the RFID chip.

According to an aspect, the invention may provide a DNA collection device that employs, for example, a drug that inhibits tyrosine kinases (e.g., tyrosine kinase inhibitor (TKI)) or other similar inhibitors. The inhibitors may be contained in strips (e.g., cloth strips) treated with the inhibitor, which strips may be housed in individual DNA collection chambers. The TSI or TKI inhibitor may extend the lifetime of collected DNA specimens.

According to an aspect, the invention may provide a specimen collection cylinder and plunger assembly, with a mesh treated) (e.g., with TKI or TSI) to avoid cross contamination mounted to the cylinder and a RFID chip mounted to the mesh. The plunger and cylinder may be employed to suction DNA into the mesh. The DNA specimen in the mesh may be readily transferred to a DNA specimen storage device (e.g., an evidence bag) while avoiding risk of cross contamination. The DNA specimen storage device may be RFID matched with the sample to ensure correct identification of the DNA sample.

According to an aspect, the invention may provide a DNA collection device that collects DNA specimens related to investigations of sexual crimes. The DNA collection device may comprise a probe with a tip (which may be snap-off and may be treated with TKI or TSI) that is configured to put into a human orifice to obtain DNA sample. The DNA specimen storage device used when investigating sexual crimes may be RFID matched with the sample to ensure correct identification of the DNA sample.

According to an embodiment, various components may form a DNA collection kit or a DNA collection system.

Various aspects of this invention will become apparent to those skilled in the art from the following detailed description of the preferred embodiments, when read in light of the accompanying drawings.

### BRIEF DESCRIPTION OF THE DRAWINGS

FIG. 1 is a schematic perspective view of a DNA collection device and kit.

FIG. 2 is a schematic perspective view similar to FIG. 1, but with an end cap shown removed.

FIG. 3 is a schematic perspective view similar to FIG. 1, but with an end cap and a DNA collection chamber shown removed.

FIG. 4 is a schematic end view of one of the DNA collection chambers.

FIG. 5 is a schematic perspective view of an alternative embodiment of a DNA collection device.

FIG. 6 is a schematic side view of an internal portion of a DNA collection device.

FIG. 7 is a schematic side view of an internal portion of a DNA collection device.

FIG. 8 is a schematic view of a pair of DNA collection strips.

FIG. 9 is a schematic side view of a DNA collection chamber.

FIG. 10 is a schematic side view of a DNA collection chamber.

FIG. 11 is a schematic view of a radio-frequency identification (RFID) chip in communication with an RFID receiving device and a transmission and storage system.

FIG. 12 is a schematic side view of a portion of a DNA collection device.

FIG. 13 is a schematic view of a DNA collection chamber in an evidence storage container.

FIG. 14 is a schematic side view of a specimen marking chamber.

FIG. 15 is a schematic side view of a portion of a DNA collection device.

FIG. 16 is a schematic perspective view of an internal portion of a DNA collection device.

FIG. 17 is a schematic perspective view of a portion of a specimen collection cylinder of an alternate embodiment of a DNA collection device.

FIG. 18 is a schematic end view of a portion of a specimen collection chamber.

FIG. 19 is a schematic end view of a mesh employed in a specimen collection chamber.

FIG. 20 is a schematic perspective view of a portion of a specimen collection cylinder assembly of an alternate embodiment of a DNA collection device.

FIG. 21 is a schematic side view of a specimen collection plunger.

FIG. 22 is a schematic perspective view of a plunger plate.

FIG. 23 is a schematic perspective view of a specimen collection cylinder assembly.

FIG. 24 is a schematic view similar to FIG. 23 but with the specimen collection plunger in a different position.

FIG. 25 is a schematic view similar to FIG. 23 but with the specimen collection plunger in a different position.

FIG. 26 is a schematic view similar to FIG. 23 but with the specimen collection plunger in a different position.

FIG. 27 is a schematic view similar to FIG. 23 but with the specimen collection plunger and the collection mesh in a different position.

FIG. 28 is a schematic perspective view of an alternate embodiment of a DNA collection device.

FIG. 29 is a schematic side view of a DNA collection chamber.

FIG. 30 is a schematic side view of a DNA collection device.

FIG. 31 is a schematic side view similar to FIG. 30, but with a DNA collection chamber separated from the DNA collection device.

FIG. 32 is a schematic side view of a probe strip support.

FIG. 33 is a schematic end view of a DNA collection chamber.

FIG. 34 is a schematic side view of a DNA collection strip on a probe strip support.

FIG. 35 is a schematic side view of a portion of a DNA collection chamber.

FIG. 36 is a schematic side view of a portion of a DNA collection chamber.

FIG. 37 is a schematic end view of a probe tip base.

FIG. 38 is a schematic side view of a stem.

FIG. 39 is a schematic side view of a portion of a DNA collection device.

### DETAILED DESCRIPTION OF THE PREFERRED EMBODIMENTS

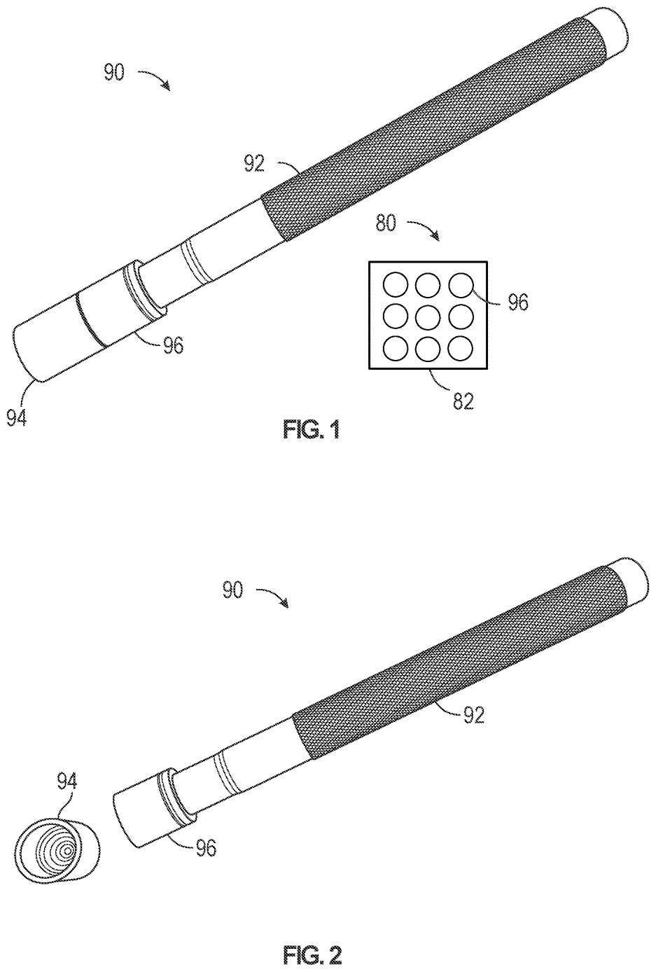

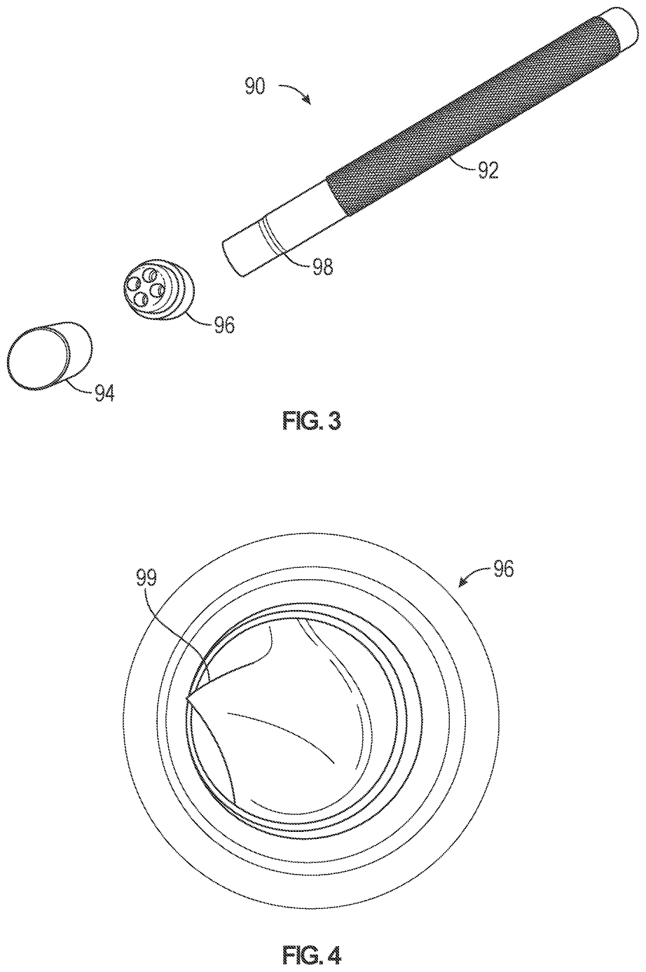

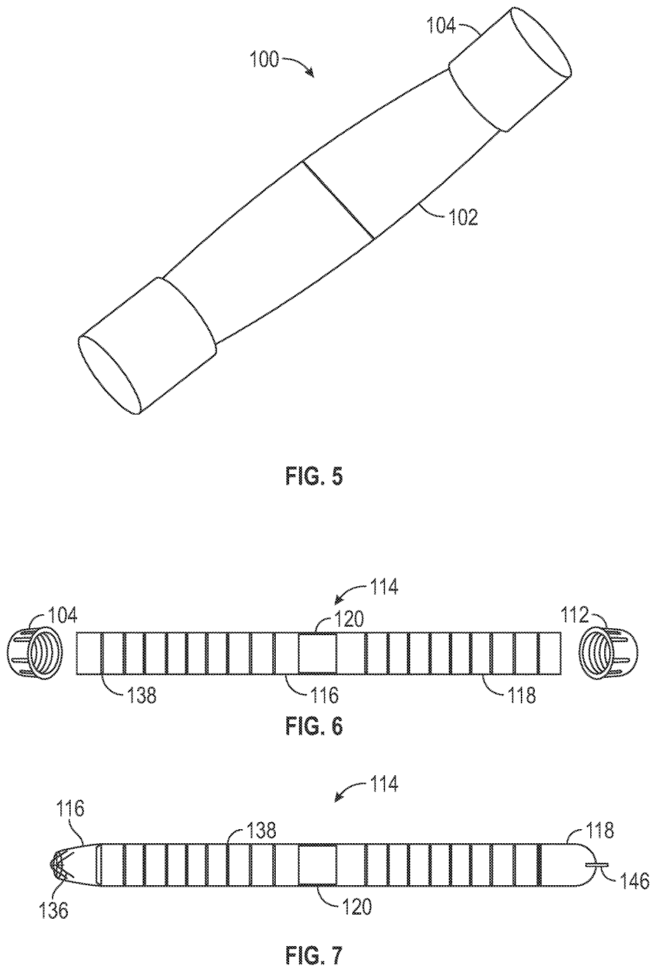

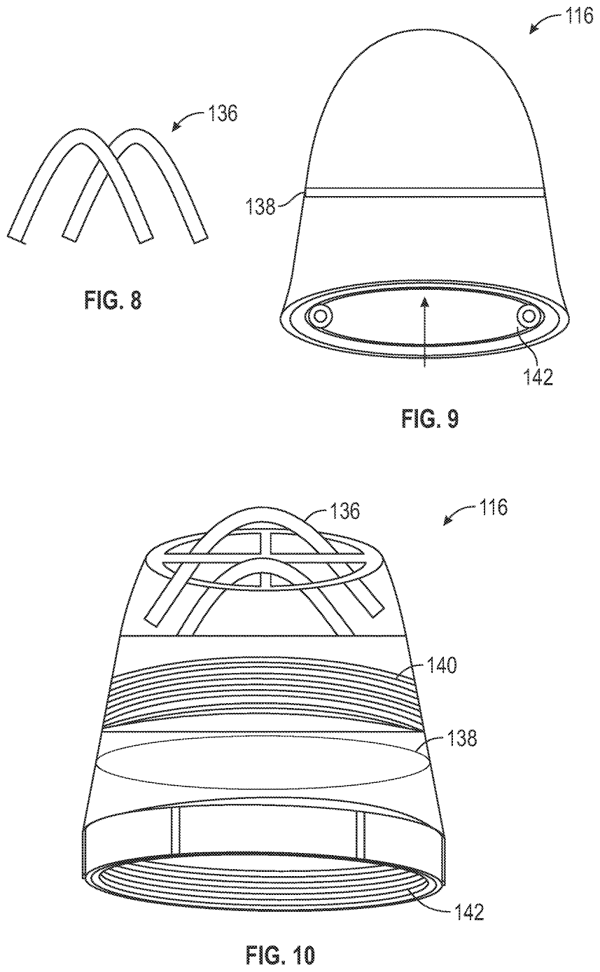

Referring to FIGS. 1-4 (in view of FIGS. 5-16) illustrate an example of a housing 92 of a deoxyribonucleic acid (DNA) collection device 90 and kit is illustrated. The housing 92 may be made of metal or plastic and may be relatively small and slender to allow for convenience in keeping the DNA collection device 90 in a pocket or portable case. As a non-limiting example, the housing 92 may be about 12-13 centimeters long and have a diameter of about 1-2 centimeters. The housing 92 may contain a master electronics device (as described below and illustrated in FIGS. 5-16). A DNA end cap 94 is removably mounted to a DNA collection chamber 96, which is removably mounted to the DNA collection device 90. The DNA end cap 94 may be used to seal and protect the DNA collection chamber 96 when the DNA collection device 90 is not in use; this may be secured with a friction fit or by other means. An O-ring 98 may seal between the DNA collection chamber 96 and the housing 92 to seal-out contaminants from the inside of the housing 92 and to releasably secure the DNA collection chamber 96 to the housing 92. A DNA specimen collection assembly 99 may be mounted in the DNA collection chamber 96. The DNA specimen collection assembly 99 may include a DNA collection chamber having elements like those discussed below and illustrated in FIGS. 5-16 (e.g., treated DNA collection strips, seal-RFID chips (or RFID chips not mounted in a seal), evidence container storage, etc.). The DNA end cap 94 and the DNA collection chamber 96 may be mounted based on pressure snap-on and pressure pop-off for assembly and removal from the DNA collection device 90. The DNA collection device 90 may be part of a DNA collection kit 80, with one or more spare DNA collection chambers 96 stored in a container 82 (in such a manner so that the spare DNA collection chamber 96 are not contaminated), and one DNA collection chamber 96 mounted on the housing 92. When the first DNA sample is taken, the first DNA collection chamber 96 is placed in an evidence storage container (such as discussed below and illustrated in FIG. 13) and a second DNA collection chamber 96 is taken from the container 82 and placed on the housing 92 for future used in taking a DNA sample. If not being immediately used to take a sample, the DNA end cap 94 is placed over the second DNA collection chamber 96 to keep out contaminants.

An example illustrated in FIGS. 5-16, in view of FIGS. 1-4, will now be discussed. The housing 102 is illustrated having the DNA end cap 104 removably mounted to one end and a marking end cap 112 removably mounted at the other end of the housing 102. Within the housing 102 is an inner assembly 114, which includes a plurality of DNA collection chambers 116 (ten in the example illustrated in FIGS. 6, 7 and 15) adjacent to the DNA end cap 104, a plurality of marker members 118 (ten in the example illustrated in FIGS. 6, 7 and 15), and a master electronics device 120.

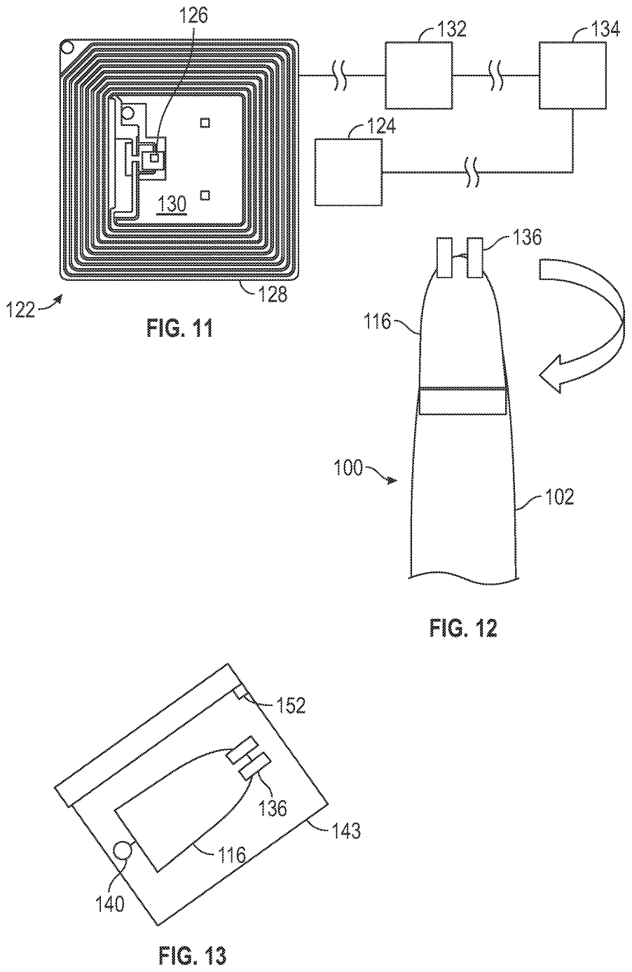

The master electronics device 120 may include a master radio-frequency identification (RFID) tag 122, a satellite chip 124 and other electronics. The master RFID tag 122 may include a control chip 126 and an antenna coil 128 on a substrate 130 (as best seen in FIG. 11). The RFID tag 122 may identify specifics related to the particular DNA collection device 100 in which it is located. The satellite chip 124 may record GPS coordinates relating to where a particular DNA sample was taken. This may include time and date information as well. The master RFID tag 122 and satellite chip 124 may communicate (e.g., wirelessly) to a signal receiving device 132, which in turn may communicate with a transmission and storage system 134. The transmission and storage system 134 may be a local computer (e.g., a laptop, tablet, cellphone) or may be cloud storage connected to the internet. This allows for easy access to the data collected as well as backup of the information. The transmission and storage system 134 may also be connected to, for example, criminal databases to allow for the matching of the DNA sample with known DNA of a criminal in the databases.

The DNA collection chamber 116 may include one or more DNA collection strips 136. Having more than one collection strip 136 allows for simultaneous collection of DNA samples (i.e., employing two DNA collections strips 136 (as illustrated in FIGS. 7, 8, 10, 12 and 13) for each sample taken allows for one DNA collection strip 136 to be tested while the other is kept for a backup in case it is ever needed in the future. The DNA collection strips 136 are brought into contact with the area of the evidence for which a DNA sample is desired. The DNA collection strips 136 may be made of, for example, cotton, and treated with a tyrosine Kinase inhibitor (TKI), which inhibits enzymes responsible for activation of proteins by signal transduction cascades, such as for example acalabrutinib or imatinib. Alternatively, the DNA collection strips 136 may be treated with tyrosinase inhibitor (TSI), such as for example arbutin. The use of the inhibitors on the DNA collection strips 136 may extend the life span of the DNA samples taken without contaminating the DNA sample. This allows for the DNA samples to be stored for a long time and still be analyzed at a much later date.

Each of the DNA collection chambers 116 may also include a seal-RFID chip 138, a first attachment feature 140 and a second attachment feature 142. Each seal-RFID chip 138 provides a seal between that DNA collection chamber 116 and the adjacent DNA collection chamber 116, and also stays with its corresponding DNA collection chamber 116 for RFID identification purposes. In this way, at a later time, the particular DNA sample taken with the particular DNA collection chamber 116 can be accurately identified by the RFID chip 138. The first attachment feature 140 connects to the DNA end cap 104, if it is at that time adjacent to the DNA end cap 104, or to an adjacent one of the DNA collection chambers 116, if not. The first attachment feature 140 may be, for example, threads (FIGS. 10, 12), or may be another type of connection (FIG. 9) (e.g., snap-off connection). The second attachment feature 142 may be, for example, threads or may be another type of connection (e.g., snap-off connection. No matter which type of connections are used for the attachment features 140, 142, the seal-RFID chip 138 and the attachment features 140, 142 combine to assure that each of the DNA collection chambers 116 is sealed from the other DNA collection chambers 116. Accordingly, the risk of cross contamination between DNA samples taken with different DNA collection chambers 116 is eliminated, even though the DNA collection device 100 contains multiple DNA collection chambers 116.

Once a particular DNA collection chamber 116 has been used to collect a DNA sample and removed from the DNA collection device, it may be placed in an evidence storage container (e.g., an evidence bag) 143, which can then be closed to assure that the DNA sample is not contaminated. The evidence storage container 143 may have areas on its outer surface that allow for information to be written and may include RFID chips 152 that match the particular evidence storage container 143 to a particular DNA collection kit from which it came.

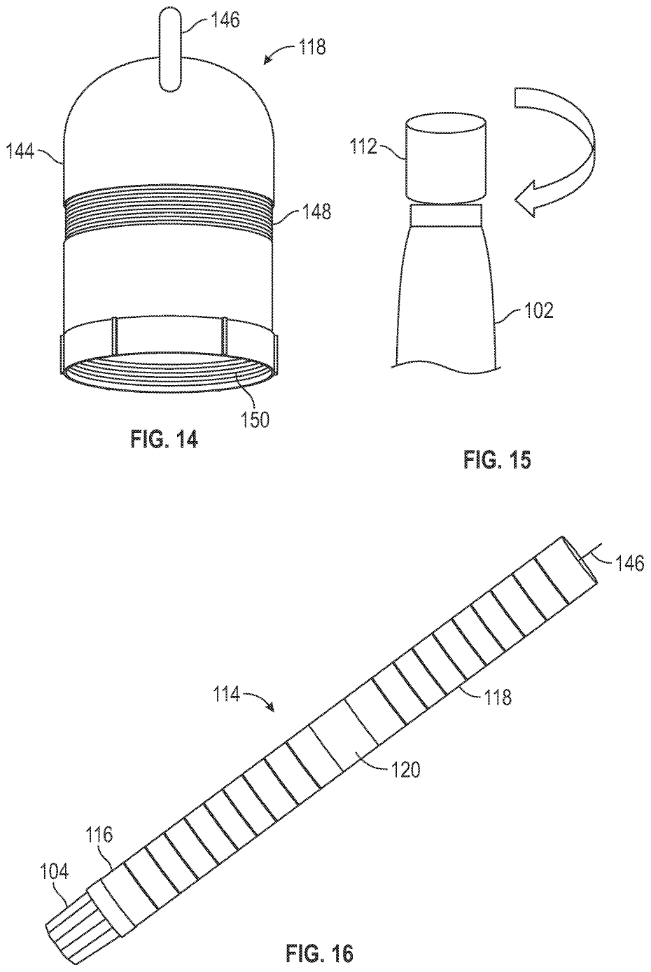

Each of the marker members 118 includes a marker solution that may include, for example TKI or TSI, and chlorophyll dye, and which may be in a marker chamber 144. The marker solution may be released into a pressure activated tip 146 for marking during DNA collection. The marking may be used to identify where the particular DNA sample was taken from on the evidence being examined without contaminating the DNA on the evidence. Each of the marker members 118 may also include a first marking attachment feature 148 and a second marking attachment feature 150. The first marker attachment feature 148 connects to the marking end cap 112, if it is at that time adjacent to the marking end cap 112, or to an adjacent one of the marker members 118, if not. The first marking attachment feature 148 may be, for example, threads (FIGS. 14, 15), or may be another type of connection (e.g., snap-off connection). The second marking attachment feature 150 may be, for example, threads or may be another type of connection (e.g., snap-off connection. No matter which type of connections are used for the marking attachment features 148, 150, the marker chamber 144 and walls of the marker member 118 combine to assure that each of the marker members 118 is sealed from the others.

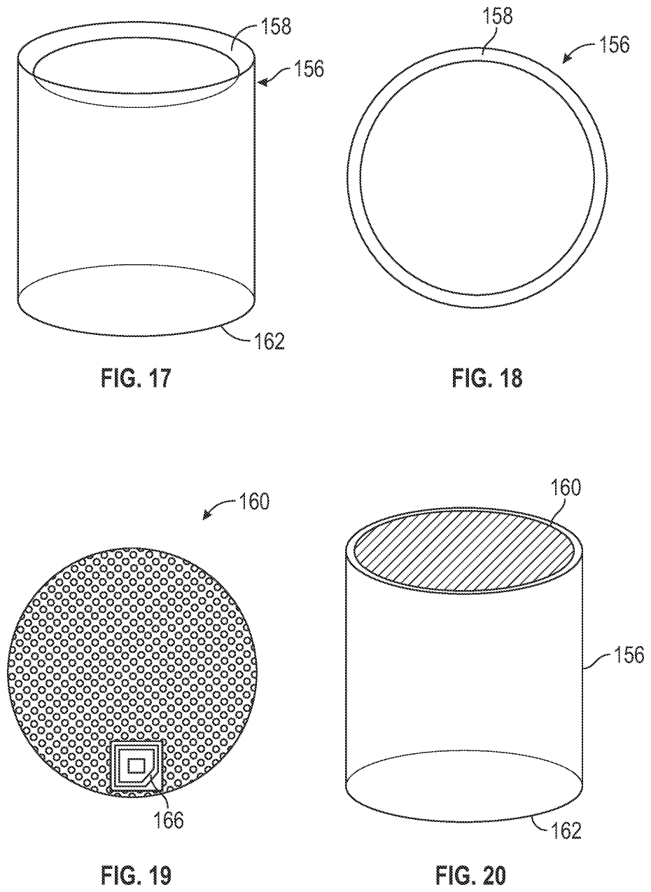

FIGS. 17-27 illustrate an example of an alternate DNA collection device 154. The DNA collection device 154 includes a specimen collection cylinder 156 that includes two open ends. The first end (rim) 158 includes a recess for receiving a collection mesh 160, and the second end 162 is open for receiving a specimen collection plunger 164. The collection mesh 160 is treated with TKI or TSI (as discussed in the examples above) and includes a RFID chip 166, which may be employed as discussed in the examples above. The specimen collection plunger 164 includes a stem 168, with a handle 170 attached to a first end and a plunger plate assembly 172 attached to a second end. The plunger plate assembly 172 may include a surface 174 for pushing on the collection mesh 160 and an annular recess 176 for receiving a seal (O-ring) 178, which seals against an inside surface of the specimen collection cylinder 156.

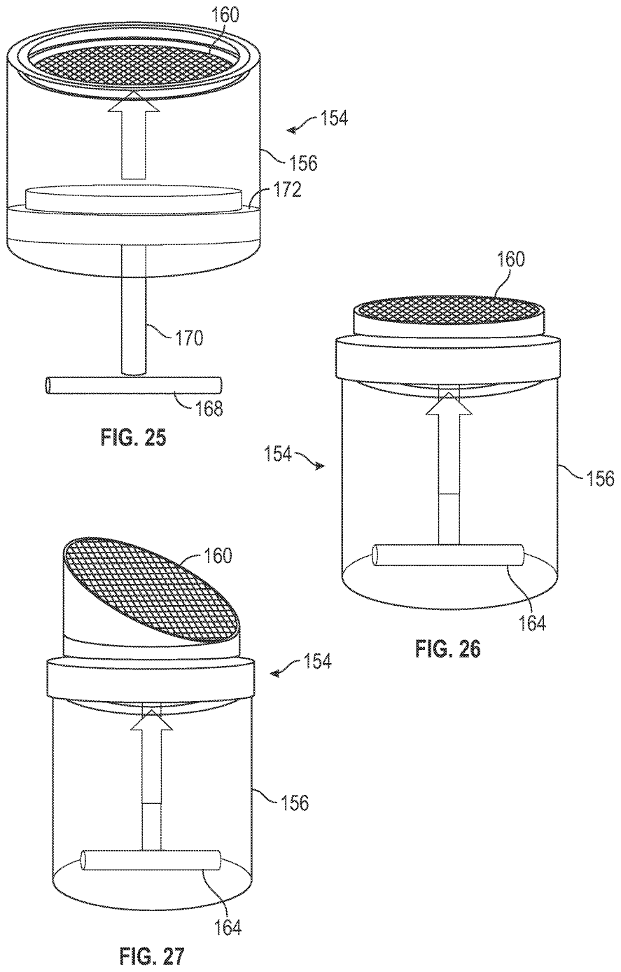

Collection of the DNA sample employing the DNA collection device 154 will now be described. The collection mesh 160 is placed against the evidence from which one wished to obtain a DNA sample, with the specimen collection plunger 164 located in the specimen collection cylinder 156 such that the surface 174 is adjacent to the collection mesh 160 (FIG. 23). One then pulls on the handle 170 in the direction of the arrows show in FIGS. 23 and 24 until the specimen collection plunger 164 is in the position shown in FIG. 24. This motion creates a suction that pulls the DNA sample into the collection mesh 160. One then moves the DNA collection device away from the evidence and pushes on the handle in the direction shown by the arrow in FIG. 25 until the specimen collection plunger 164 is in the position illustrated in FIG. 26. One then pushes on the handle further in the direction illustrated in FIGS. 26 and 27 until the collection mesh 160 releases from the rim 158 (shown in FIG. 27). The collection mesh 160 may be released into an evidence bag, such as that illustrated and discussed above relative to FIG. 13, which evidence bag may include an RFID tag.

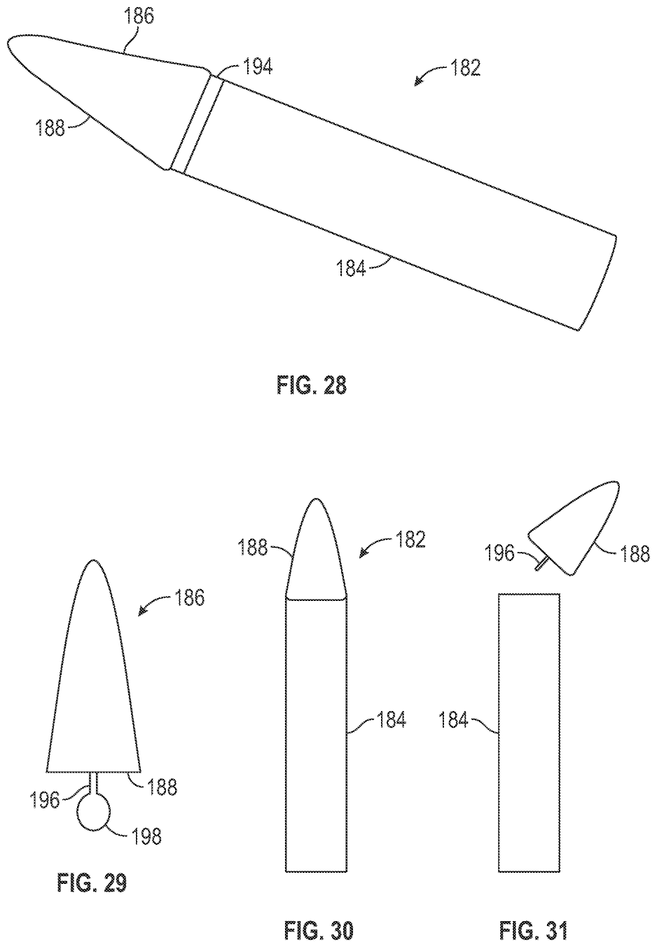

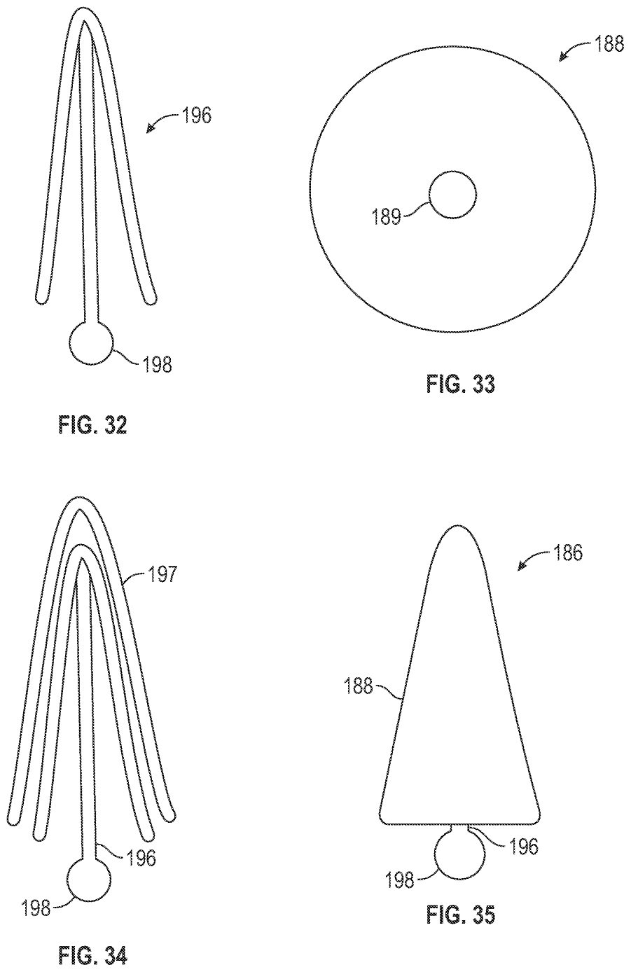

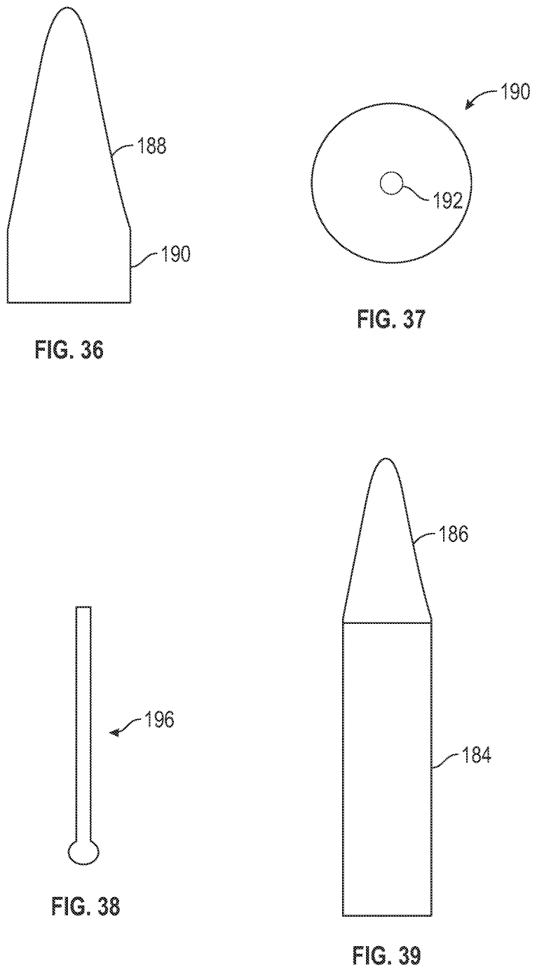

FIGS. 28-39 illustrate an example of an alternate DNA collection device 182. The DNA collection device 182 is sized and shaped to be inserted into human orifices, for example to be used for investigations of sexual assault. As such, the DNA collection device 182 includes a long slender cylindrical stem 184 upon which a probe tip assembly 186 is mounted. The probe tip assembly 186 includes a hollow conically shaped DNA collection chamber 188, having a sample hole 189, connected with a probe tip base 190, having a center hole 192, a seal-RFID chip assembly 194, a probe strip support 196 (FIGS. 32 and 38 illustrate alternative shapes) and a DNA collection strip 197. The DNA collection strip may be treated with TKI or TSI as discussed relative to the examples above. The DNA collection strip 197 mounts on the probe strip support 196, which is fitted through the center hole 192 of the probe tip base 190 and held by a spherical end 198 that has a larger diameter than the center hole 192. The DNA collection strip 197 is engaged with the sample hole 189 at the narrow end of the conical DNA collection chamber 188. The seal-RFID chip assembly 194 is secured to the DNA collection chamber 188. The probe tip assembly 186 is releasably mounted to the stem 184, with, for example, a twist-off or snap-off connection.

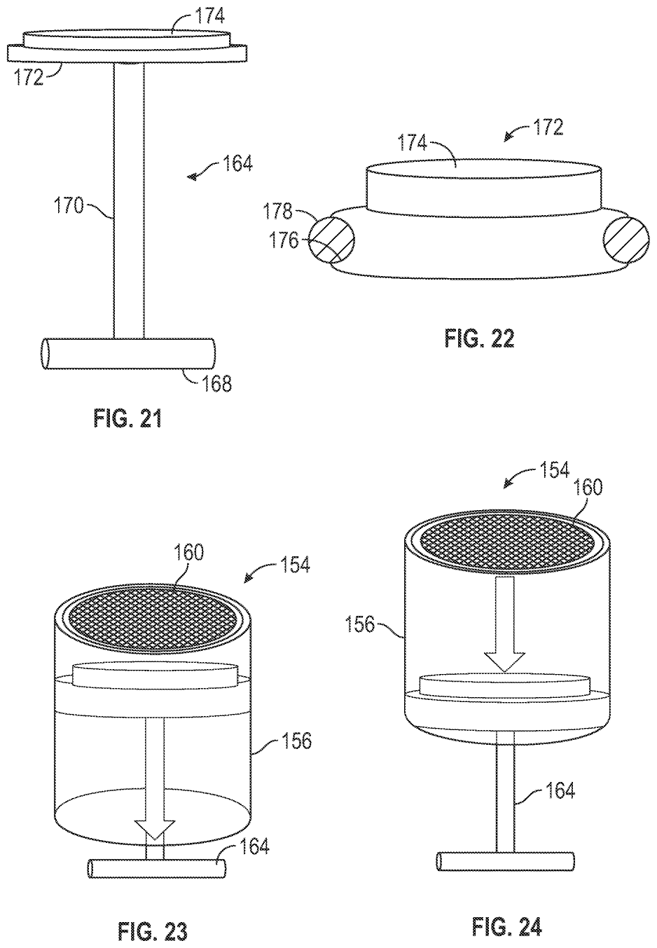

To take a DNA sample with the DNA collection device 182, one inserts the DNA collection device 182 (FIGS. 28, 30, 39) into, for example, an orifice of a human body from which one wishes to obtain a DNA sample. The DNA collection strip 197 picks up the DNA sample, at which point the DNA collection device 182 is removed from the orifice. The DNA collection chamber 188 and seal-RFID chip assembly 194 are removed from the stem 184 and placed into an evidence bag, such as that illustrated and discussed above relative to FIG. 13. An example of a snap-off configuration is illustrated in FIGS. 30 and 21, where, after taking the DNA sample, the DNA collection chamber 188 and seal-RFID chip assembly 194 are snapped-off by breaking the spherical end 198 off from the probe strip support 196.

The principle and mode of operation of this invention have been explained and illustrated in its preferred embodiment. However, it must be understood that this invention may be practiced otherwise than as specifically explained and illustrated without departing from its spirit or scope.

1. A DNA collection system comprising:
a DNA collection device including:
an elongated hollow housing having a first end and an opposed second end; and
a plurality of DNA collection chambers mounted in a row in the housing, adjacent to the first end, each of the DNA collection chambers including a DNA collection component, and the collection component of each of the DNA collection chambers sealed from the other DNA collection components of the other DNA collection chambers, wherein each of the DNA collection chambers is removably mounted to adjacent DNA collection members;
a plurality of marker members mounted in a row in the housing, adjacent to the second end, with the marker members configured to mark where a DNA sample is taken, wherein each of the marker members is removably mounted to adjacent marker members, each of the marker members including a pressure activated tip for releasing a marking chemical.

2.-5. (canceled)

6. The DNA collection system of claim 1 wherein the DNA collection component in each of the DNA collection chambers is treated with a TKI or a TSI or both.

7. The DNA collection system of claim 1 wherein the DNA collection device further includes a master electronic communication tag mounted in the housing, or the DNA collection device includes a master electronic communication device and each of the DNA collection chambers includes an electronic communication device.

8. (canceled)

9. The DNA collection system of claim 7 wherein the DNA collection device further includes a satellite communication chip mounted thereto, wherein the satellite communication chip is configured to determine at least one of a GPS coordinate, time and date for where and when a DNA sample is taken by one of the DNA collection chambers.

10.-12. (canceled)

13. The DNA collection system of claim 1 wherein the DNA collection component in each of the DNA collection chambers is two cloth strips protruding from the corresponding DNA collection chamber, wherein each of the cloth strips receives a DNA sample when the DNA sample is being taken.

14. The DNA collection system of claim 1 further including an evidence storage container having a storage RFID chip mounted thereto, wherein the evidence storage container is configured to receive one of the DNA collection chambers after a DNA sample is taken.

15.-16. (canceled)

17. A DNA collection device comprising:
a specimen collection cylinder that is hollow, with a first end and an opposed second end; and
a collection mesh mounted to the specimen collection cylinder adjacent to the first end, wherein the collection mesh is treated with at least one of a TKI and a TSI.

18. The DNA collection device of claim 17 further including a specimen collection plunger having a plunger plate assembly mounted in and sealed against an inner surface of the specimen collection cylinder, the specimen collection plunger configured to draw a vacuum in the specimen collection cylinder to draw a DNA sample into the collection mesh when the plunger plate assembly is moved away from the collection mesh.

19. The DNA collection device of claim 17 further comprising an elongated probe stem having an end, and a probe tip assembly removably mounted to the end and including a hollow conically shaped DNA collection chamber having a sample hole at a narrow end spaced from the stem, a DNA collection component in the DNA collection chamber adjacent to the sample hole configured to collect a DNA sample through the sample hole.

20. The DNA collection device of claim 18 wherein the specimen collection plunger is configured to eject the collection mesh from the specimen collection cylinder when pressed against the collection mesh.

21. The DNA collection device of claim 17 further including a RFID chip mounted to the collection mesh.

22.-26. (canceled)

27. A kit for collecting DNA, the kit comprising:
a DNA collection device including:
an elongated hollow housing having a first end;
a master electronic communication tag mounted to the housing; and
a DNA collection chamber removably mounted to the first end and including a DNA collection component mounted thereto, wherein the DNA collection component is treated with at least one of a TKI and a TSI.

28. The kit of claim 27 further comprising:
a container; and
at least a second DNA collection chamber located in the container, wherein the at least the second DNA collection chamber includes a second DNA collection component mounted thereto that is treaded with at least one of the TKI and the TSI, and wherein the at least the second DNA collection chamber is configured to mount on the first end of the housing.

29. The kit of claim 28 wherein the DNA collection component is s pair of cloth strips protruding from the DNA collection chamber and treated with at least one of the TKI and the TSI.

30. The kit of claim 28 wherein each of the DNA collection chamber and the at least the second DNA collection chamber includes a RFID an electronic communication tag.

31. The kit of claim 27 wherein the DNA collection component is s pair of cloth strips protruding from the DNA collection chamber and treated with at least one of the TKI and the TSI.

32. The kit of claim 27 wherein the DNA collection device further includes a DNA end cap removably mountable over the DNA collection chamber and configured to seal the DNA collection chamber from contaminants.

33. The kit of claim 27 wherein the master electronic communication tag is a master RFID tag and the DNA collection chamber includes a RFID tag.

34. The kit of claim 27 wherein the DNA collection device further include a satellite communication chip mounted thereto, wherein the satellite communication chip is configured to determine at least one of a GPS coordinate, time and date for where and when a DNA sample is taken by the DNA collection chamber.

35. The kit of claim 27 further including a transmission and storage system for receiving and storing data related to a DNA sample taken by the DNA collection chamber.
